SEQUENCE LISTING (12 sequences)

SEQ ID NO: 1 - DNA, 23 bp, Artificial
  primer
tgcgaagttttgtgactgaaaca

SEQ ID NO: 2 - DNA, 23 bp, Artificial
  primer
attcttggagcattcagcctttc

SEQ ID NO: 3 - DNA, 23 bp, Artificial
  primer
tacaacggtgtttgtgcagatcc

SEQ ID NO: 4 - DNA, 23 bp, Artificial
  primer
ggaatgtgacagcaagatctcgt

SEQ ID NO: 5 - DNA, 23 bp, Artificial
  primer
tcataattacccctccgacactg

SEQ ID NO: 6 - DNA, 23 bp, Artificial
  primer
ggagagagaccttggtcgaagag

SEQ ID NO: 7 - DNA, 23 bp, Artificial
  primer
tgaagatgcacactgtgtcctac

SEQ ID NO: 8 - DNA, 23 bp, Artificial
  primer
gtctactgctggatccataccct

SEQ ID NO: 9 - DNA, 23 bp, Artificial
  primer
cagctactacacggaatcaccaa

SEQ ID NO: 10 - DNA, 23 bp, Artificial
  primer
cttggtagattggattgctgctg

SEQ ID NO: 11 - DNA, 23 bp, Artificial
  primer
ttactgaagaggtccgactgtgc

SEQ ID NO: 12 - DNA, 23 bp, Artificial
  primer
ccacagaattcaggtcaatgagg

(19) United States
(12) Patent Application Publication
(10) Pub. No.: US 2018/0271930 A1
(43) Pub. Date: Sep. 27, 2018

(54) METHOD FOR RAISING POULTRY

(71) Applicant: Kaneka Corporation, Osaka (JP)

(72) Inventors: Kazuyoshi Yajima, Osaka (JP); Kan Sato, Tokyo (JP)

(73) Assignee: Kaneka Corporation, Osaka (JP)

(21) Appl. No.: 16/003,975

(22) Filed: Jun. 8, 2018

Related U.S. Application Data
(63) Continuation of application No. PCT/JP2016/086360, filed on Dec. 7, 2016.

(30) Foreign Application Priority Data
Dec. 8, 2015 (JP) 2015-239348

(51) Int. Cl. A61K 38/06; A23K 20/147; A23K 50/75; A61K 9/00

(52) U.S. Cl. CPC A61K 38/063; A61K 9/0056; A23K 50/75; A23K 20/147

(57) ABSTRACT

A method for raising poultry includes feeding poultry with a feed comprising oxidized glutathione during a period between hatching and 24 to 168 hours after the hatching.

### TECHNICAL FIELD

One or more embodiments of the present invention relate to a method for raising poultry such as chickens and an initial-phase feed composition for poultry.

One or more embodiments of the present invention also relate to a method for increasing muscle mass of poultry.

One or more embodiments of the present invention further relate to a method for reducing expression levels of a factor that inhibits skeletal muscle differentiation, a factor that inhibits skeletal muscle growth, or a factor that promotes skeletal muscle growth differentiation in poultry chicks, and a method for reducing expression levels of myostatin and/or myogenin.

### BACKGROUND

The present inventors previously elucidated that it is possible to inhibit skeletal muscle cell differentiation so as to promote skeletal muscle cell growth by giving an initial feed containing insulin, an insulin-like factor, or an antioxidant to neonatal broiler chicks, thereby improving meat production by grown chickens or increasing their body weights (Non Patent Literature 1).

Meanwhile, glutathione is a peptide consisting of three amino acids which are L-cysteine, L-glutamic acid, and glycine. Glutathione is present not only in the human body but also in many organisms such as other animals, plants, and microorganisms. Glutathione is an important compound for the living body in terms of active oxygen scavenging, detoxification, amino acid metabolism, and so on.

Glutathione exists in vivo in the form of reduced glutathione which is formed when a thiol group of an L-cysteine residue is reduced to result in formation of an SH group (hereinafter also referred to as “GSH”) or oxidized glutathione which is formed when a thiol group of an L-cysteine residue is oxidized to result in formation of a disulfide bond between two glutathione molecules (hereinafter also referred to as “GSSG”).

There are references reporting that glutathione is mixed in feeds for raising animals. For example, Patent Literature 1 discloses that glutathione may be added as an antioxidant to a poultry feed containing a high-moisture substance. Patent Literature 2 discloses that glutathione may be added as an antioxidant to a feed for companion animals, which contains pyruvate in an amount effective for treatment of kidney injury. Patent Literature 3 discloses that glutathione is added as an active ingredient of a feed for reducing oxidative stress in animals of the family Felidae. Patent Literature 4 discloses that glutathione is a sleep inducer effective for animals such as livestock and pets.

Patent Literature 5 suggests that glutathione is used as an antioxidant to be added to a functional food or beverage, and the use of glutathione and vitamins C and E in combination enables preventing aging of muscle tissues.

However, none of Patent Literature 1 to 5 discloses using oxidized glutathione as glutathione.

### CITATION LIST

### Patent Literature

Patent Literature 1: JP Patent Publication No. H11-506617 (1999) A
Patent Literature 2: JP Patent Publication No. 2013-515780 A
Patent Literature 3: JP Patent Publication No. 2013-82713 A
Patent Literature 4: JP Patent Publication No. 2008-56628 A
Patent Literature 5: JP Patent Publication No. 2003-267992 A
Patent Literature 6: International Publication WO 2013/002317

### Non Patent Literature

Non Patent Literature 1: General and Comparative Endocrinology 175 (2012), 457-463

There is a demand to increase poultry weights in order to efficiently produce meat of poultry including chickens. There are a variety of conventional feeds provided for increasing poultry weights, however, such feeds are not always satisfactory. Patent Literature 1 to 4 disclose that glutathione may be added to a feed composition, but fail to consider whether or not poultry weights can be increased using such composition.

### SUMMARY

One or more embodiments of the present invention provide a breeding method effective for increasing the poultry weight and a method for increasing muscle mass of poultry. Moreover, one or more embodiments of the present invention provide a method for reducing expression levels of myostatin and/or myogenin, which promotes skeletal muscle cell differentiation, in order to promote the growth of skeletal muscle cells in neonatal poultry chicks. Furthermore, one or more embodiments of the present invention provide an initial-phase feed composition for poultry, which is suitable for these methods.

A surprising finding is that feeding neonatal poultry chicks with a feed comprising oxidized glutathione is beneficial in increasing muscle mass of grown poultry to increase their body weights and in reducing expression levels of myostatin and/or myogenin, which promotes skeletal muscle cell differentiation in skeletal muscle of initial-phase chicks, resulting in the growth of skeletal muscle cells. Specifically, one or more embodiments of the present invention encompass the following inventions.

(1) A method for raising poultry, comprising a step of feeding poultry with a feed comprising oxidized glutathione during a period between hatching and 24 to 168 hours after hatching.
(2) A method for increasing muscle mass of poultry, comprising a step of feeding poultry with a feed comprising oxidized glutathione during a period between hatching and 24 to 168 hours after hatching.
(3) A method for reducing expression levels of myostatin and/or myogenin in skeletal muscles of poultry, comprising a step of feeding poultry with a feed comprising oxidized glutathione during a period between hatching and 24 to 168 hours after hatching.
(4) An initial-phase feed composition for poultry, comprising oxidized glutathione, which is an initial-phase feed composition for poultry and, for example, poultry in a development stage between hatching and 24 to 168 hours after hatching.

According to the method in (1) above, poultry suitable for meat production can be efficiently produced.

According to the method in (2) above, muscle mass of poultry can be efficiently increased.

According to the method in (3) above, it is possible to reduce expression levels of myostatin and/or myogenin, which promote skeletal muscle cell differentiation, in skeletal muscles of poultry chicks. As a result, skeletal muscle cell growth can be promoted.

Feeding neonatal chicks with the initial-phase feed composition for poultry in (4) above, can improve an increase in the body weight of poultry.

One or more embodiments of the present invention further encompass the following inventions.

(5) Oxidized glutathione or a feed composition comprising oxidized glutathione for feeding poultry during a period between hatching and 24 to 168 hours after hatching.
(6) Oxidized glutathione or a feed composition comprising oxidized glutathione for feeding poultry during a period between hatching and 24 to 168 hours after hatching to increase muscle mass of the poultry.
(7) Oxidized glutathione or a feed composition comprising oxidized glutathione for feeding poultry during a period between hatching and 24 to 168 hours after hatching to reduce expression levels of myostatin and/or myogenin in skeletal muscles of the poultry during the period.
(8) Use of oxidized glutathione for producing a feed composition for feeding poultry during a period between hatching and 24 to 168 hours after hatching.
(9) Use of oxidized glutathione for producing a feed composition for feeding poultry during a period between hatching and 24 to 168 hours after hatching to increase muscle mass of the poultry.
(10) Use of oxidized glutathione for producing a feed composition for feeding poultry during a period between hatching and 24 to 168 hours after hatching to reduce expression levels of myostatin and/or myogenin in skeletal muscles of the poultry during the period.
(11) Use of oxidized glutathione or a feed composition comprising oxidized glutathione for feeding poultry during a period between hatching and 24 to 168 hours after hatching.
(12) Use of oxidized glutathione or a feed composition comprising oxidized glutathione for feeding poultry during a period between hatching and 24 to 168 hours after hatching to increase muscle mass of the poultry.
(13) Use of oxidized glutathione or a feed composition comprising oxidized glutathione for feeding poultry during a period between hatching and 24 to 168 hours after hatching to reduce expression levels of myostatin and/or myogenin in skeletal muscles of the poultry during the period.

The methods according to (1) to (3) above are typically non-medical methods.

The initial-phase feed composition according to (4) above is typically a non-medical initial-phase feed composition.

The oxidized glutathione or feed composition according to (5) to (7) above may be for medical or non-medical use.

The feed compositions according to (8) to (10) above may be for medical or non-medical use.

The use according to any one of (11) to (13) above is typically non-medical use.

The term “medical” used herein refers to a medical purpose for poultry.

The present description includes part or all of the contents as disclosed in the description and/or drawings of Japanese Patent Application No. 2015-239348, which is a priority document of the present application.

According to one or more embodiments of the present invention, a breeding method effective for improving an increase in the poultry weight, a method for increasing muscle mass of poultry, and a method for reducing expression levels of myostatin and/or myogenin in initial-phase poultry chicks are provided. According to one or more embodiments of the present invention, an initial-phase feed composition for poultry, which is suitable for these methods, is also provided.

### BRIEF DESCRIPTION OF DRAWINGS

FIGS. 1A to 1D illustrate graphs showing the expression of genes related to skeletal muscle cell differentiation in superficial pectoral muscles upon administration of each feed composition in Examination 1 (n=6). FIG. 1A shows relative values of the MyoD expression level, FIG. 1B shows relative values of the myogenin expression level, FIG. 1C shows relative values of the myostatin expression level, and FIG. 1D shows relative values of the IGF-I expression level. A relative value of the expression level of each factor is expressed as an average±standard deviation of values obtained by dividing the expression levels of each factor by the expression level of RPS9. Each different symbol denotes a significant difference in the Tukey-kramer multiple comparison test (P<0.05).

FIGS. 2A to 2D illustrate graphs showing the expression levels of genes related to skeletal muscle cell differentiation upon the addition of GSSG during the skeletal muscle cell differentiation period in Examination 3 (n=6). FIG. 2A shows relative values of the MyoD expression level, FIG. 2B shows relative values of the myostatin expression level, FIG. 2C shows relative values of the IGF-I expression level, and FIG. 2D shows relative values of the Pax7 expression level. A relative value of the expression level of each factor is expressed as an average±standard deviation of values obtained by dividing the expression levels of each factor by the expression level of RPS9. Each different symbol denotes a significant difference in the Tukey-kramer multiple comparison test (P<0.05).

FIGS. 3A to 3C illustrate graphs showing the expression of genes related to skeletal muscle cell differentiation in superficial pectoral muscles upon administration of each feed composition in Examination 4 (n=4). FIG. 3A shows relative values of the myogenin expression level, FIG. 3B shows relative values of the myostatin expression level, and FIG. 3C shows relative values of the Pax7 expression level. A relative value of the expression level of each factor is expressed as an average±standard deviation of values obtained by dividing the expression levels of each factor by the expression level of RPS9.

### DETAILED DESCRIPTION OF EMBODIMENTS

### 1. Subject Poultry

The “poultry” according to one or more embodiments of the present invention are not particularly limited as long as domesticated birds are used. Examples of poultry that can be used include chickens, quails, turkeys, ducks, geese, and rice ducks. The poultry includes both poultry for meat collection and poultry for egg collection.

The poultry according to one or more embodiments of the present invention include, as chickens, broilers that are chickens for meat collection, layer chickens that are chickens for egg collection, and breeder chickens thereof. Specific examples of broiler breeds include Chunky (also known as ROSS) and COBB.

### 2. Feed Containing Oxidized Glutathione

Oxidized glutathione (GSSG) is a substance formed by two molecules of reduced glutathione (GSH, N—(N-γ-L-glutamyl-L-cysteinyl)glycine) bound via a disulfide bond.

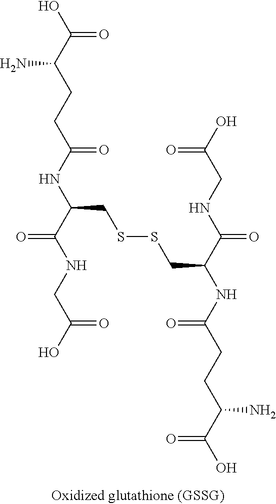 [SMILES: N[C@@H](CCC(=O)N[C@@H](CSSC[C@H](NC(=O)CC[C@H](N)C(=O)O)C(=O)NCC(=O)O)C(=O)NCC(=O)O)C(=O)O]

According to one or more embodiments of the present invention, oxidized glutathione (GSSG) may encompass various forms of GSSG including salts formed with acids or bases with free GSSG which are not bound to other substances, hydrates thereof, and mixtures thereof. Free GSSG is expressed by the above formula. In addition, GSSG may be in a state of being present in cells that produce GSSG or in a state of being contained in a pulverized product of such cells. Similarly, according to one or more embodiments of the present invention, reduced glutathione (GSH) may encompass various forms of GSH including salts formed with acids or bases with free GSH which are not bound to other substances, hydrates thereof, and mixtures thereof. Further, GSH may be in a state of being present in cells that produce GSH or in a state of being contained in a pulverized product of such cells.

A feed used in one or more embodiments of the present invention may comprise reduced glutathione as well as oxidized glutathione. However, it may be possible that the mass of oxidized glutathione is relatively greater than the mass of reduced glutathione in the feed, and that the feed comprises substantially no reduced glutathione. In one or more embodiments, in a feed used in one or more embodiments of the present invention, the total mass of oxidized glutathione (mass in terms of free oxidized glutathione) relative to the total mass of oxidized glutathione and reduced glutathione (total mass in terms of free oxidized glutathione and free reduced glutathione) may be 70% by mass or more, 80% by mass or more, 90% by mass or more, 95% by mass or more, 98% by mass or more, or 100% by mass.

A GSSG salt used herein is not particularly limited as long as it is at least one type of salt acceptable as a feed, such as, an ammonium salt, a calcium salt, a magnesium salt, a sodium salt, or a lithium salt. However, at least one type of salt selected from ammonium salts, calcium salts, and magnesium salts may be used. As disclosed in Patent Literature 6, an ammonium salt, a calcium salt, or a magnesium salt of GSSG in the solid form may be used because it is a little deliquescent, easy to handle, and highly water-soluble. Such salt can be obtained in the solid form by bringing GSSG into contact with an aqueous medium selected from water and/or a water-soluble medium with heating to 30° C. or higher under the presence of a substance that can produce at least one selected from an ammonium ion, a calcium cation, and a magnesium cation as described in Patent Literature 6. The heating temperature is not particularly limited as long as it is 30° C. or higher. However, it may be 33° C. or higher, 35° C. or higher, or 40° C. or higher. The upper limit thereof is not particularly limited. However, for example, it may be 80° C. or lower, 70° C. or lower, or 60° C. or lower. It may be possible that the heating temperature is within a range of 53° C. to 60° C. for industrial-scale production. The aqueous medium may be used alone, or two or more types of aqueous media may be used in combination, if appropriate. However, using a mixture of water and a water-soluble medium is recommended. In such case, the water functions as a rich solvent for oxidized glutathione, and the water-soluble medium functions as a poor solvent. The volume of the water-soluble medium may be, for example, about 1 to 1000 parts by volume, about 5 to 500 parts by volume, about 10 to 100 parts by volume, or about 12 to 50 parts by volume with respect to 10 parts by volume of the water. Examples of the water-soluble medium include alcohols (e.g., methanol, ethanol, propanol, butanol, and ethylene glycol) and ketones (e.g., acetone and ethyl methyl ketone). Examples of a GSSG salt obtained by the above method include a monoammonium salt of GSSG, a hemicalcium salt or a monocalcium salt of GSSG, or a hemimagnesium salt or a monomagnesium salt of GSSG.

The content of oxidized glutathione in a feed used in one or more embodiments of the present invention is not particularly limited. However, the content of oxidized glutathione, in terms of free oxidized glutathione, may be 0.001% by mass or more, or 0.01% by mass or more, and it may be 1.0% by mass or less, 0.1% by mass or less, 0.07% by mass or less, or 0.05% by mass or less with respect to the total amount of the feed (based on the wet weight).

Other components of a feed used in one or more embodiments of the present invention are feed components used in ordinary poultry feeds, which may include grain (e.g., corn, barley, wheat, and milo), oil meal (e.g., soybean meal, cottonseed meal, rapeseed meal, sesame meal, or linseed meal), and food manufacturing residues (e.g., bran, wheat bran, sake lees, or corn production residues), fermentation by-products, dairy by-products, slaughter-plant by-products, and fats and oils (derived from, for example, animals and plants).

Before feeding poultry with each of the above feed components including oxidized glutathione, it may be possible to provide a feed composition that has been mixed with such component in advance. However, the present invention is not limited thereto. A composition comprising one of the above components or a plurality of compositions comprising a plurality of the components may be given to poultry.

The feed may be in the form of liquid, paste, solid, powder, granules, or in any other form.

A feed used in one or more embodiments of the present invention may be provided in the form of an initial-phase feed composition for poultry. The “initial-phase feed composition for poultry” is a feed composition suitable for feeding poultry chicks in the initial growth phase and specifically chicks during a period between hatching and approximately 168 hours after hatching, and it is also referred to as a “prestarter feed.”

### 3. Feeding

Each method according to one or more embodiments of the present invention is characterized in that poultry are fed with the feed comprising oxidized glutathione during a period between hatching and 24 to 168 hours after hatching. In one or more embodiments, the period ranges from hatching to 24 to 120 hours after hatching. In one or more embodiments, the period ranges from hatching to 40 hours or longer after hatching. It may be possible to feed poultry with an oxidized glutathione-containing feed alone during the period. However, the present invention is not limited thereto. It is possible to feed poultry additionally with a feed free of oxidized glutathione. In a case in which chicks to be shipped after hatching are used, supply of the oxidized glutathione-containing feed can be started after receipt of the chicks. After the start of feed supply, it may be possible to give the oxidized glutathione-containing feed every day until the end of the period. Here, for example, hatched chicks will be received within 24 hours after hatching.

It is possible to give a feed to poultry by an ordinary feed supply method for feeding/raising poultry using, for example, a paper mat inside a chick guard, a feedbox, or an automatic feeder. For example, the poultry's feed intake during the period is 15 to 17 g at 1 day old and 20 to 22 g at 2 days old. Poultry at 3 days old or older are fed ad libitum.

The total amount of glutathione consumed by one chick during the period can be set to, for example, 1 mg or more, or 10 mg or more in terms of free glutathione. Although the upper limit is not limited, it can be set to, for example, 300 mg or less, or 100 mg or less. By setting the total amount of oxidized glutathione consumed during the period within the above range, it is possible to increase muscle mass of grown poultry, thereby increasing body weights. In addition, expression levels of myostatin and/or myogenin, which promote skeletal muscle cell differentiation, are reduced in skeletal muscles of initial-phase chicks, thereby making it possible to allow skeletal muscle cell growth. In order to adjust the total amount of oxidized glutathione consumed within the above range by the end of the period, it is possible to feed poultry chicks with an oxidized glutathione-containing feed at once or in divided portions (for example, 2 or 3 times). However, it may be possible to feed chicks continuously with an oxidized glutathione-containing feed, and specifically, on a daily basis after hatching of chicks (receipt of chicks in a case in which hatched chicks are received for use) until the end of the period.

According to the method of one or more embodiments of the present invention, poultry are fed with the feed comprising oxidized glutathione during the period between hatching and 24 to 168 hours after hatching or the above-described period, and then, the poultry may be fed with an ordinary feed and raised until their body weights reach body weights for shipment (for example, 3 kg for broilers). The term “ordinary feed” refers to a feed comprising no or substantially no oxidized glutathione. The poultry's age in days upon shipment is usually 42 days old or older after hatching. However, one or more embodiments of the present invention can be applied to all poultry at 21 days old or older. In other words, according to the method of one or more embodiments of the present invention, it is possible to increase muscle mass of poultry, thereby enabling efficient meat production including shortening of the age in days upon shipment and increasing of the body weight for shipment.

### 4. Reduction of Myostatin Expression Levels

One or more embodiments of the present invention relate to a method for reducing expression levels of myostatin in skeletal muscles of poultry during a period between hatching and 24 to 168 hours after hatching. According to this method, it is not necessary to reduce the expression level of myostatin throughout the period. It is necessary to reduce the expression level of myostatin to a level lower than that in the case of a feed free of oxidized glutathione in a stage when the total intake of oxidized glutathione reaches the effective amount, for example, when at least the endo of the period.

In one or more embodiments of the present invention, the expression level of myostatin is an expression level determined by the following measurement method. Specifically, the expression level can be determined by extracting total RNA from skeletal muscles or skeletal muscle cells, performing reverse transcription, and quantitatively determining an amplification product of myostatin by quantitative PCR using the obtained cDNA as a template. These operations may be conducted using existing reagent kits. The amount of an amplification product of myostatin can be expressed as a value divided by the amount of an amplification product of RPS9 obtained in the same manner for conversion to total RNA present in a sample. A myostatin DNA amplification primer set that can be used is, but is not limited to, a set of a sense primer comprising the nucleotide sequence of SEQ ID NO: 3 and an antisense primer comprising the nucleotide sequence of SEQ ID NO: 4. A RPS9DNA amplification primer set that can be used is, but is not limited to, a set of a sense primer comprising the nucleotide sequence of SEQ ID NO: 1 and an antisense primer comprising the nucleotide sequence of SEQ ID NO: 2. A real-time PCR method can be used for quantitative PCR. However, other methods can be used, if appropriate.

### 5. Reduction of Myogenin Expression Levels

One or more embodiments of the present invention relate to a method for reducing expression levels of myogenin in skeletal muscles of poultry during a period between hatching and 24 to 168 hours after hatching. According to this method, it is not necessary to reduce the expression level of myogenin throughout the period. It is necessary to reduce the expression level of myogenin to a level lower than that in the case of a feed free of oxidized glutathione in a stage when the total intake of oxidized glutathione reaches the effective amount, for example, when at least the end of the period.

In one or more embodiments of the present invention, the expression level of myogenin can be determined as in the case of the method for determining the expression level of myostatin except that quantitative PCR is performed using a myogenin DNA amplification primer set instead of a myostatin DNA amplification primer set. A myogenin DNA amplification primer set that can be used is, but is not limited to, a set of a sense primer comprising the nucleotide sequence of SEQ ID NO: 5 and an antisense primer comprising the nucleotide sequence of SEQ ID NO: 6.

### Examples

Hereinafter, one or more embodiments of the present invention are described in detail with reference to specific examples. However, the specific examples below do not limit the scope of the present invention.

### Examination 1

### Examination Method

Newly hatched broiler chicks of the Chunky breed (Ross 308, Ross Breeders Ltd.) were tested. A control group and a test group were separately determined (3 groups×2 levels of additive concentration), fed with a feed composition for 3 days, and slaughtered. Here, the three-day feeding means that chicks were received at 0 day old and fed with any of test teed compositions until 72 hours after the receipt. Blood and skeletal muscles were collected to observe gene expression levels of MyoD, myogenin, myostatin, and IGF-I (insulin-like growth factor-I).

MyoD and myogenin belong to the MyoD family and are factors for inducing differentiation of muscle satellite cells into myoblasts. In addition, myostatin is a factor that inhibits myoblast growth. IGF-I is a factor that promotes myoblast growth and differentiation formation into muscle fibers. In the initial growth phase of chickens, muscle satellite cell growth and differentiation into myoblasts continue until the hatched chickens become 3 days old. When the chickens become 4 days old or older, muscle satellite cell growth and differentiation into myoblasts are suspended. In order to increase skeletal muscle cells so as to increase muscle mass of grown adult chickens, it is considered beneficial to inhibit the expression of MyoD, myogenin, or myostatin in skeletal muscle cells until chickens become about 3 days old.

A method for determining gene expressions of the above factors in skeletal muscles is described below.

Total RNA was extracted from a homogenized skeletal muscle sample using a commercially available total RNA extraction reagent (TRIZOL Thermo Fisher Scientific). Next, cDNA was prepared from Total RNA using a commercially available reverse transcriptase (M-MLV reverse transcriptase Thermo Fisher Scientific). Real-time PCR was performed to amplify DNA of RPS9, IGF-I, myostatin, MyoD, or myogenin using the cDNA as a template. Real-time PCR was performed by adding cDNA, a primer set, and other necessary reagents to wells of a 96-well PCR plate, preheating the plate using a thermal cycler at 95° C. for 3 minutes, followed by 35 cycles of steps of denaturation, annealing, and elongation in a repetitive manner, and measurement of absorbance after the elongation step for each cycle. The expression level of each gene was designated as a relative value obtained by dividing the determined amount of an amplification product of IGF-I, myostatin, MyoD, or myogenin by the amount of an amplification product of RPS9. A primer set for amplification of each gene and PCR conditions used for PCR are listed in the table below.

TABLE 1

| | | Denaturation/Annealing/ |
|---|---|---|
| Gene | Primer set | Elongation conditions |
| RPS9 | Sense: TGCGAAGTTTTGTGACTGAAACA (SEQ ID NO: 1) | 94° C. 30 sec./63° C. 45 sec./ |
| | Antisense: ATTCTTGGAGCATTCAGCCTTTC (SEQ ID NO: 2) | 72° C. 30 sec. |
| Myostatin | Sense: TACAACGGTGTTTGTGCAGATCC (SEQ ID NO: 3) | 94° C. 30 sec./64° C. 45 sec./ |
| | Antisense: GGAATGTGACAGCAAGATCTCGT (SEQ ID NO: 4) | 72° C. 30 sec. |
| Myogenin | Sense: TCATAATTACCCCTCCGACACTG (SEQ ID NO: 5) | 94° C. 30 sec./65° C. 50 sec./ |
| | Antisense: GGAGAGAGACCTTGGTCGAAGAG (SEQ ID NO: 6) | 72° C. 60 sec. |
| IGF-1 | Sense: TGAAGATGCACACTGTGTCCTAC (SEQ ID NO: 7) | 94° C. 30 sec./64° C. 45 sec./ |
| | Antisense: GTCTACTGCTGGATCCATACCCT (SEQ ID NO: 8) | 72° C. 30 sec. |
| MyoD | Sense: CAGCTACTACACGGAATCACCAA (SEQ ID NO: 9) | 94° C. 30 sec./65° C. 50 sec./ |
| | Antisense: CTTGGTAGATTGGATTGCTGCTG (SEQ ID NO: 10) | 72° C. 30 sec. |

An ammonium salt was used as oxidized glutathione (GSSG). The GSSG contained 95.6% by mass of oxidized glutathione, and the content of reduced glutathione mixed therein was 0.1% by mass or less.

A feed composition, for which the level of oxidized glutathione (GSSG) to be added was set to 0.01% by mass or 0.1% by mass, is a composition prepared by adding GSSG at 0.01% by mass or 0.1% by mass to a composition (ME3,100 kcal/kg, CP21) containing corn, soybean meal, corn gluten meal, fat and oil, calcium carbonate (powder), tricalcium phosphate, dietary salt, DL-methionine, L-lysine chloride, choline chloride, and vitamins/minerals in accordance with the requirements of NRC (National Research Council, U.S.A.). The amount of GSSG used herein was determined in terms of the amount of free oxidized glutathione. In all the following Examinations, the amounts of GSSG are expressed in terms of the amount of free oxidized glutathione.

In the above and following descriptions, the term “ME” used refers to “metabolic energy,” the term “CP” refers to “crude protein,” and the terms “CP21” and “CP18” mean a crude protein content of 21% by mass and a crude protein content of 18% by mass, respectively.

Chicks were fed with each feed composition on a paper mat, and they were given the feed using a feedbox after day 3 and later. The poultry's feed intake during the period was set to 15 to 17 g at 1 day old and 20 to 22 g at 2 days old. Poultry at 3 days old or older were fed ad libitum.

### Examination Timing

Early June (feeding test), analysis from June to July

### Results

Table 2 shows body weights at 3 days old. The body weights at 3 days old showed a tendency to increase with the addition of glutathione at 0.01% by mass. However, it was not a significant increase.

TABLE 2
Body weights of initial-phase chicks fed with each
additive-containing feed composition (g, n = 6)

| | |
|---|---|
| Weight at 0 day old | 37.5 ± 2.7 |
| Weight at 3 days old (g) | |
| Control group | 73.7 ± 4.8 |
| GSSG addition group (0.1%) | 72.8 ± 5.6 |
| GSSG addition group (0.01%) | 78.1 ± 4.0 |

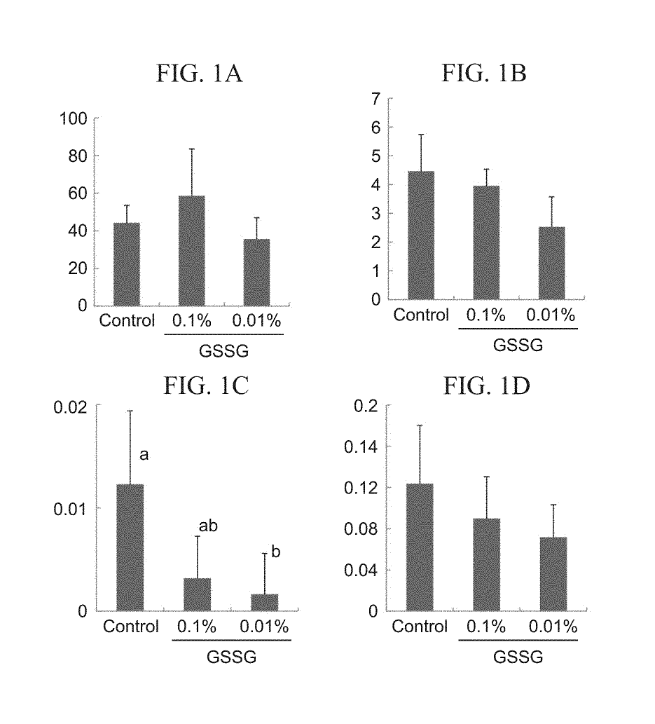

FIGS. 1A to 1D show measurement results of gene expression levels of the skeletal muscle cell differentiation factors at 3 days old. There were significant decreases in the myogenin expression level (FIG. 1B) and the myostatin expression level (FIG. 1C) in the GSSG addition group as compared to the control group. The above suggest a possibility that GSSG can inhibit skeletal muscle cell differentiation in neonatal chicks.

### Examination 2-1

### Examination Method

Newly hatched broiler chicks (Ross Breeders Ltd.) were fed with a feed composition for 3 days. The feed composition contained GSSG at a level of 0.01% by mass which was thought to be appropriate based on Examination 1. Thereafter, the composition was replaced by an ordinary feed composition of the same type and the chicks were raised from 21 days old to 28 days old after hatching. It was examined whether or not feeding could influence body weight increase or meat productivity.

### Examination Timing

October (feeding test), analysis in November

### Test Group

Two groups, which were a control group and a GSSG addition group (0.01% by mass), were set.

In each group, 6 males and 6 females were tested. Chicks received at 0 day old were fed with a test feed composition (initial-phase feed composition) during 72 hours after the receipt. Thereafter, the chicks were raised with a feed composition (ME3,100 kcal/kg, CP21) free of GSSG, which was the same as that used in Examination 1, until they became 21 days old or 28 days old. Their body weights, breast meat weights, leg meat weights, and liver weights were measured.

As the initial-phase feed composition, the feed composition comprising 0.01% by mass of GSSG used in Examination 1 was used for the GSSG addition group, and the feed composition (ME3,100 kcal/kg, CP21) free of GSSG used in Examination 1 was used for the control group.

Chicks were fed with each feed composition on a paper mat, and they were given the feed using a feedbox after day 3 and later. The poultry's feed intake during the period was set to 15 to 17 g at 1 day old and 20 to 22 g at 2 days old. Poultry at 3 days old or older were fed ad libitum.

An ammonium salt was used as oxidized glutathione (GSSG). The GSSG contained 95.6% by mass of oxidized glutathione, and the content of reduced glutathione mixed therein was 0.1% by mass or less.

### Results

There was no significant difference between the GSSG group and the control group. However, the body weights at 21 days old and 28 days old showed a tendency to increase as compared to the control group (Table 3).

TABLE 3
Performance of breeding chicks fed with the
GSSG-containing prestarter feed composition (n = 6)

| | 21 days old | | | |
|---|---|---|---|---|
| | | GSSG | 28 days old | |
| | Control | addition | Control | GSSG |
| | group | group | group | addition group |
| Weight (g) | 908 ± 65 | 933 ± 65 | 1450 ± 149 | 1500 ± 149 |
| Breast meat (g) | 102 ± 11 | 118 ± 4 | 192 ± 16 | 186 ± 15 |
| Leg meat (g) | 130 ± 7 | 136 ± 6 | 200 ± 13 | 206 ± 12 |
| Liver (g) | — | — | 38 ± 3 | 37 ± 2 |

### Examination 2-2

### Examination Method

Newly hatched broiler chicks (Ross 308, Ross Breeders Ltd.) were fed with a feed composition for 3 days as in the case of Examination 2-1. Thereafter, the composition was replaced by an ordinary feed composition of the same type and the chicks were raised until they became 40 days old. It was examined whether or not feeding could influence the body increase at 40 days old, meat yield, and feed conversion ratio.

### Examination Timing

January to February

### Test Group

Two groups, which were a control group and a GSSG addition group (0.01% by mass) were set.

In each group, 6 males and 6 females were tested. Chicks received at 0 day old were fed with a test feed composition (initial-phase feed composition) during 72 hours after the receipt. Thereafter, the chicks were raised with a feed composition (ME3,100 kcal/kg, CP21) free of GSSG, which was the same as that used in Examination 1, until they became 21 days old. Thereafter, the chicks were raised until they became 40 days old with a feed composition (ME3,200 kcal/kg, CP18) free of GSSG. The body weight was measured, and the meat yield and feed conversion ratio were determined.

As the initial-phase feed composition, the 0.01% by mass GSSG-containing feed composition used in Examination 1 was used in the GSSG addition group, and the feed composition (ME3,100 kcal/kg, CP21) free of GSSG used in Examination 1 was used for the control group.

The above-described feed composition (ME3,200 kcal/kg, CP18) contained corn, milo, soybean meal, chicken meal, fish powder, fat and oil, calcium carbonate (powder), tricalcium phosphate, dietary salt, DL-methionine, L-lysine chloride, choline chloride, and vitamins/minerals in accordance with the NRC requirements.

An ammonium salt was used as oxidized glutathione (GSSG). The GSSG contained 95.6% by mass of oxidized glutathione, and the content of reduced glutathione mixed therein was 0.1% by mass or less.

Chicks were fed with each feed composition on a paper mat, and they were given the feed using a feedbox after day 3 and later. The poultry's feed intake during the period was set to 15 to 17 g at 1 day old and 20 to 22 g at 2 days old. Poultry at 3 days old or older were fed ad libitum.

The term “feed conversion ratio” refers to an amount (g) of a feed required for a body weight increase of 1 g.

The term “meat yield” refers to a proportion of meat excluding bones and fats with respect to the body weight. The meat yield can be determining by performing slaughtering and butchering, collecting meat alone, and weighing the collected meat.

### Results

Table 4 shows the results.

A significant body weight increase was observed in the GSSG group, and the feed conversion ratio showed a tendency to improve with a significant difference in the GSSG group (P=0.06).

TABLE 4
Performance of breeding chicks fed with the
GSSG-containing prestarter feed composition (n = 12)

| | Control group | GSSG addition group |
|---|---|---|
| Weight (g) | 2097 ± 94 | 2214 ± 79* |
| Meat yield (% by mass) | 42.0 ± 0.8 | 42.1 ± 1.1 |
| Feed conversion ratio | 1.63 ± 0.03 | 1.52 ± 0.09 |
| (mass ratio) | | |

*Significant difference as compared with the control group (P < 0.05)

### Examination 3

### Examination Method

Oxidized glutathione (GSSG) was added to a differentiation-promoting culture medium system of skeletal muscle cells collected from newly hatched broiler chicks (Ross 308, Ross Breeders Ltd.) (under culture conditions simulating the state of skeletal muscle cells of neonatal chicks), phosphorylation of signal factors was analyzed by Western blot, and the differentiation inhibition mechanism was investigated.

Expression levels of MyoD, myostatin, IGF-I, and Pax7 as genes involved in myogenesis were determined. Pax7 is a muscle satellite cell marker. A method for determining expression levels of MyoD, myostatin, and IGF-I is described in Examination 1. The expression level of Pax7 was determined by the method described in Examination 1 except that the primer set used herein was a primer set listed in the table below.

TABLE 5

| | | Denaturation/ |
|---|---|---|
| | | Annealing/ |
| | | Elongation |
| Gene | Primer set | conditions |
| Pax7 | Sense: TTACTGAAGAGGTCC | 94° C. 30 sec./ |
| | GACTGTGC (SEQ ID NO: 11) | 64° C. 45 sec./ |
| | Antisense: CCACAGAATTCA | 72° C. 30 sec. |
| | GGTCAATGAGG (SEQ ID NO: 12) | |

### Examination Timing

December, analysis in January

### Test Group

An experiment was conducted using the differentiation-promoting culture medium system of skeletal muscle cells described in Non Patent Literature 1, which is simulating the state of skeletal muscle cells of neonatal chicks. This experiment is in accordance with the method detailed in Non Patent Literature 1. The outline of the experiment is described below.

Myoblasts prepared from broiler chicks at 0 day old were seeded in a φ90-mm dish at a concentration of 5×10³/cm², a medium (i.e., a medium prepared by mixing DMEM (SIGMA-ALDRICH) and M199 (SIGMA-ALDRICH) at a ratio of 4:1 and adding FBS (fetal bovine serum: bio west) and Penicillin-Streptomycin (Pen Strep: gibco) such that their final concentrations are 10% and 1%, respectively) was added thereto, and culture was carried out in an environment at 37° C. in 5% CO². When the cells were confirmed to have become subconfluent, the cells were reseeded on a 12-well plate (Sumitomo Bakelite Co., Ltd., collagen, Type I coat). The medium was replaced by a differentiation medium (i.e., a medium prepared by adjusting the FBS concentration to 2% in the above medium) 12 hours after reseeding. Then, the experiment was initiated. When the medium was replaced by the differentiation medium, 100 μM of GSSG was added. The cells were recovered 0, 12, and 24 hours later, and the expression levels of genes involved in myogenesis were determined.

An ammonium salt was used as oxidized glutathione (GSSG). The GSSG contained 95.6% by mass of oxidized glutathione, and the content of reduced glutathione mixed therein was 0.1% by mass or less.

### Results

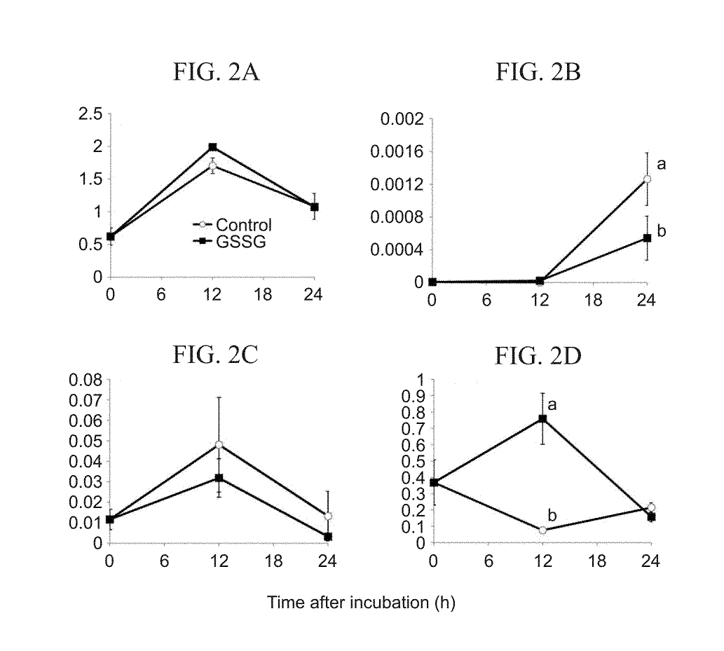

FIGS. 2A to 2D show variations in gene expression. As in the case of Examination 1, the addition of GSSG caused a significant decrease in the mRNA expression level of myostatin serving as a skeletal muscle cell growth inhibitory factor (FIG. 2B). Similarly, the expression of Pax7 serving as a marker for myocytes capable of proliferating significantly increased 12 hours after the start of culture in the GSSG addition group as compared to the control group (FIG. 2D).

### Conclusion of Examinations 1 to 3.

It was revealed that GSSG causes myocytes, which can proliferate, to increase in the differentiation induction phase (in the same situation as in the initial growth phase) through inhibition of the gene expression of myogenin and/or myostatin serving as a skeletal muscle cell growth inhibitory factor.

### Examination 4

### Examination Method

Newly hatched broiler chicks (Ross Breeders Ltd.) were tested. A control group (=additive-free group), a GSSG addition group (0.01% by mass), and a GSH addition group (0.01% by mass) each consisting of 5 males and 5 females were set and fed with a feed composition for 3 days. Here, the three-day feeding means that chicks were received at 0 day old and fed with any of test teed compositions until 72 hours after the receipt. At this timing, 2 males and 2 females in each group were slaughtered to collect blood and skeletal muscles. The gene expression levels of the MyoD family (myogenin, myostatin, and Pax7) were observed. The feed was replaced by an ordinary feed composition, and the remaining 3 males and 3 females were continuously raised until they became 35 days old. It was examined whether or not feeding could influence body weight increase or meat productivity. Statistics were analyzed using a mixed model in consideration of a gender difference and an interaction between males and females.

A method for determining gene expressions of the above factors in skeletal muscles is described above.

An ammonium salt was used as oxidized glutathione (GSSG). The GSSG contained 95.6% by mass of oxidized glutathione, and the content of reduced glutathione mixed therein was 0.1% by mass or less.

A product used as reduced glutathione (GSH) contained 99.4% by mass of reduced glutathione and 0.1%% by mass or less of oxidized glutathione.

A feed composition, for which the amount of oxidized glutathione (GSSG) added was set to 0.01% by mass, is described in Examination 1.

A feed composition, for which the amount of reduced glutathione (GSH) added was set to 0.01% by mass, was prepared by adding 0.01% by mass of GSH, instead of 0.01% by mass of GSSG, to the GSSG-containing feed composition described in detail in Examination 1.

An additive-free feed composition, which was given to the control group for 3 days after hatching, is a feed composition (ME3,100 kcal/kg, CP21) free of GSSG and GSH as in the case of Examination 1.

A feed composition (ME3,100 kcal/kg, CP21) free of GSSG and GSH was used as an ordinary feed composition which was given at least 3 days later after hatching.

Chicks were fed with each feed composition on a paper mat, and they were given the feed using a feedbox after day 3 and later. The poultry's feed intake during the period was set to 15 to 17 g at 1 day old and 20 to 22 g at 2 days old. Poultry at 3 days old or older were fed ad libitum.

### Examination Timing

Early August (feeding test), analysis in September and October

### Results

Table 6 shows the body weight measurement results for individuals raised until they became 35 days old. The body weight showed a tendency to increase in the GSSG addition group (P=0.07). Meanwhile, there was no significant difference for GSH.

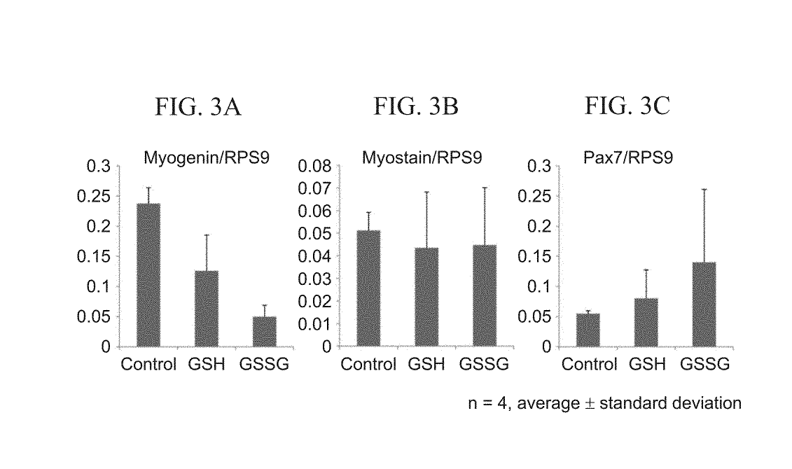

FIGS. 3A to 3C show measurement results of gene expression levels of the skeletal muscle cell differentiation factors at 3 days old. The myogenin expression level in the GSSG addition group significantly decreased as compared with the control group. A decrease in the myogenin expression level in the GSH addition group as compared with the control group was smaller than the decrease.

These results assumed that GSSG has the effect of promoting an increase in the broiler body weight, which is greater than that of GSH.

TABLE 6
Weight of chicks raised until 35 days old
after feeding of GSSG or GSH for 3 days

| | 14 days old | 35 days old |
|---|---|---|
| Control group | 486 ± 64.7 | 2054 ± 264 |
| GSH addition group | 512.7 ± 79.8 | 2052 ± 267 |
| GSSG addition group | 524.5 ± 37.0 | 2164 ± 178* |

(Unit: g, n = 6, average ± standard deviation)

### Examination 5

The effect of glutathione yeast mainly containing reduced glutathione (GSH) for improving an increase in the body weight was examined.

### Examination Method

Newly hatched broiler male chicks (Ross Breeders Ltd.) were tested. A control group (=additive-free group), a test group for which glutathione yeast was added to a feed to result in a concentration of 0.01% by mass in terms of GSH, and a test group for which glutathione yeast was added to a feed to result in a concentration of 0.1% by mass in terms of GSH were set such that each group consisted of 6 individuals or 8 individuals. Each group was fed with a feed composition for 3 days. Here, the three-day feeding means that chicks were received at 0 day old and fed with any of test teed compositions until 72 hours after the receipt. Thereafter, the feed was replaced by an ordinary feed composition, and the chicks were raised until they became 21 days old or 42 days old. It was examined whether or not feeding could influence body weight increase or meat productivity. As the ordinary feed composition, a feed composition (ME3,100 kcal/kg, CP21) was used until the chicks became 21 days old as in Examination 2-2, and then, a feed composition (ME3,200 kcal/kg, CP18) was used for raising chicks.

The feed composition, to which glutathione yeast was added to result in a concentration of 0.01% by mass or 0.1% by mass in terms of GSH, was prepared by adding glutathione yeast, instead of GSSG, to the GSSG-containing feed composition described in detail in Examination 1 to result in a predetermined content.

An additive-free feed composition, which was given to the control group for 3 days after hatching, is a feed composition (ME3, 100 kcal/kg, CP21) free of glutathione yeast as in the case of Examination 1.

Chicks were fed with each feed composition on a paper mat, and they were given the feed using a feedbox after day 3 and later. The poultry's feed intake during the period was set to 15 to 17 g at 1 day old and 20 to 22 g at 2 days old. Poultry at 3 days old or older were fed ad libitum.

The definition of meat yield and the definition of feed conversion ratio are described in Examination 2-2.

### Examination Timing

October and November (feeding test)

### Results

Tables 7 and 8 show the results.

In a case in which glutathione yeast was given to neonatal chicks and then the chicks were raised in an ordinary manner, no significant effect of increasing body weight was confirmed. It is understood that the effect of increasing body weight cannot be expected with the addition of glutathione yeast containing reduced glutathione to a feed.

TABLE 7
Weights (g) of 21-day-old chickens fed with glutathione yeast for 3 days

| Control group | 0.01% addition group | 0.1% addition group |
|---|---|---|
| 945 ± 137 | 973 ± 127 | 910 ± 90 |

n = 6, average ± standard deviation

TABLE 8
Measurement values for 42-day-old chickens fed with
glutathione yeast for 3 days (0.01% in terms of GSH)

| | Control group | Yeast addition group |
|---|---|---|
| Weight (g) | 2897 ± 94 | 2914 ± 143 |
| Meat yield (% by mass) | 42.9 ± 1.8 | 42.4 ± 1.8 |
| Feed conversion ratio | 1.73 ± 0.13 | 1.79 ± 0.20 |
| (mass ratio) | | |

n = 8, average ± standard deviation

### Examination 6

### Examination Method

Newly hatched broiler chicks (Ross Breeders Ltd.) were fed with a feed composition containing GSSG at 0.01% by mass or 0.05% by mass for 3 days. Thereafter, the composition was replaced by an ordinary feed composition of the same type and the chicks were raised until they became 21 days old after hatching. It was examined whether or not feeding could influence body weight increase.

### Examination Timing

September

### Test Group

Three groups, which were a control group, a GSSG addition group (0.01% by mass), and a GSSG addition group (0.05% by mass), were set.

In each group, 6 males and 6 females were tested. Chicks received at 0 day old were fed with a test feed composition (initial-phase feed composition) during 72 hours after the receipt. Thereafter, the chicks were raised with a feed composition (ME3,100 kcal/kg, CP21) free of GSSG, which was the same as that used in Examination 1, until they became 21 days old. Their body weights were measured.

A feed composition used as an initial-phase feed composition, for which the level of GSSG was set to 0.01% by mass or 0.1% by mass, is a composition prepared by adding GSSG at 0.01% by mass or 0.1% by mass to a composition (ME3,100 kcal/kg, CP21) containing corn, soybean meal, corn gluten meal, fat and oil, calcium carbonate (powder), tricalcium phosphate, dietary salt, DL-methionine, L-lysine chloride, choline chloride, and vitamins/minerals in accordance with the NRC requirements. The feed composition (ME3,100 kcal/kg, CP21) free of GSSG used in Examination 1 was used for the control group.

Chicks were fed with each feed composition on a paper mat, and they were given the feed using a feedbox after day 3 and later. The poultry's feed intake during the period was set to 15 to 17 g at 1 day old and 20 to 22 g at 2 days old. Poultry at 3 days old or older were fed ad libitum.

An ammonium salt was used as GSSG. The GSSG contained 95.6% by mass of oxidized glutathione, and the content of reduced glutathione mixed therein was 0.1% by mass or less.

### Results

Table 9 shows the body weight measurement results for each test group at 21 days old. The body weights at 21 days old in the GSSG addition group (0.05% by mass) were significantly greater than those in the control group.

In addition, the body weights at 21 days old in the GSSG addition group (0.01% by mass) showed a tendency to be greater than those in the control group.

TABLE 9

| | Control group | GSSG 0.01% | GSSG 0.05% |
|---|---|---|---|
| 21-day-old weight (g) | 919 ± 50 | 961 ± 84 | 986 ± 67* |

n = 12, average ± standard deviation
*Significant difference as compared with the control group (P < 0.05)

### Examination 7

### Examination Method

Newly hatched broiler chicks (Ross Breeders Ltd.) were fed with a feed composition with a blending rate of GSSG of 0.05% by mass until they became 3, 7, or 14 days old after hatching. Thereafter, in the case of feeding until they became 3 or 7 days old, the composition was replaced by an ordinary feed composition of the same type and the chicks were raised until 14 days old after hatching. It was examined whether or not feeding could influence body weight increase.

### Examination Timing

September

### Test Group

Four groups, which were a control group, a test group 1 for which a feed containing 0.05% by mass of GSSG was added until the chicks became 3 days old, a test group 2 for which a feed containing 0.05% by mass of GSSG was added until the chicks became 7 days old, and a test group 3 for which a feed containing 0.05% by mass of GSSG was added until the chicks became 14 days old, were set.

In each group, 6 males and 6 females were tested. A test feed composition (initial-phase feed composition) was given to chicks received at 0 day old until they became 3 days old (72 hours after hatching) (test group 1), until they became 7 days old (168 hours after hatching) (test group 2), or until they became 14 days old (336 hours after hatching) (test group 3) for raising. In the test groups 1 and 2, the chicks were then raised with a feed composition (ME3,100 kcal/kg, CP21) free of GSSG, which was the same as that used in Examination 1, until they became 14 days old. The chicks were raised until they became 14 days old, and then, their body weights were measured. The feed composition (ME3,100 kcal/kg, CP21) free of GSSG used in Examination 1 was used for the control group. In the control group the chicks were raised until they became 14 days old and their body weights were measured.

In the test groups 1 to 3, a feed composition used as an initial-phase feed composition, for which the level of GSSG to be added was set to 0.05% by mass, is a composition prepared by adding GSSG at 0.05% by mass to a composition (ME3,100 kcal/kg, CP21) containing corn, soybean meal, corn gluten meal, fat and oil, calcium carbonate (powder), tricalcium phosphate, dietary salt, DL-methionine, L-lysine chloride, choline chloride, and vitamins/minerals in accordance with the NRC requirements.

Chicks were fed with each feed composition on a paper mat, and they were given the feed using a feedbox after day 3 and later. The poultry's feed intake during the period was set to 15 to 17 g at 1 day old and 20 to 22 g at 2 days old. Poultry at 3 days old or older were fed ad libitum.

An ammonium salt was used as GSSG. The GSSG contained 95.6% by mass of oxidized glutathione, and the content of reduced glutathione mixed therein was 0.1% by mass or less.

### Results

Table 10 shows the body weight measurement results for the control group and test groups 1 to 3 at 14 days old.

In the test group 1, for which the GSSG-containing feed was given until the chicks became 3 days old, and the test group, for which the GSSG-containing feed was given until the chicks became 7 days old, the body weights were confirmed to have increased at 14 days old as compared to the control group. Meanwhile, in the test group 3, for which the GSSG-containing feed was given until the chicks became 14 days old, the body weights at 14 days old were not significantly different from those in the control group. It was suggested that chicks may be fed with the GSSG-containing feed until they become 7 days old or younger.

TABLE 10

| | | feeding of GSSG | | |
|---|---|---|---|---|
| | Control group | until | until | until |
| | (without GSSG) | 3 days old | 7 days old | 14 days old |
| 14-day-old | 365 ± 21 | 393 ± 17* | 391 ± 24 | 351 ± 47 |
| weight (g) | | | | |

n = 12, average ± standard deviation
*Significant difference as compared with the control group (P < 0.05)

All publications, patents and patent applications cited in the present description are incorporated herein by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A method for raising poultry, comprising feeding poultry with a feed comprising oxidized glutathione during a period between hatching and 24 to 168 hours after the hatching.

2. The method according to claim 1, wherein the poultry has an increased muscle weight, as compared to a muscle weight of poultry fed with a feed without oxidized glutathione.

3. The method according to claim 1, wherein the poultry has reduced expression levels of myostatin and/or myogenin in skeletal muscles, as compared to expression levels of myostatin and/or myogenin in skeletal muscles of poultry fed with a feed without oxidized glutathione.

4. The method according to claim 1, wherein the poultry are selected from the group consisting of chickens, quails, turkeys, ducks, geese, and rice ducks.

5. The method according to claim 1, wherein a total amount of the oxidized glutathione fed to the poultry is 1 mg or more, wherein the oxidized glutathione is calculated as free glutathione.

6. The method according to claim 1, wherein a total amount of the oxidized glutathione fed to the poultry is 10 mg or more, wherein the oxidized glutathione is calculated as free glutathione.

7. The method according to claim 1, wherein the feeding is performed during a period between hatching and 24 to 120 hours after the hatching.

8. The method according to claim 1, wherein the feeding is performed during a period between hatching and 40 to 168 hours after the hatching.
